(19) United States
(12) Patent Application Publication
(10) Pub. No.: US 2015/0008174 A1
(43) Pub. Date: Jan. 8, 2015

(54) HG SENSOR USING ANISOTROPIC AU NANOPARTICLES AND RELATED WATER REMEDIATION

(71) Applicant: University of Central Florida Research Foundation, Inc., Orlando, FL (US)

(72) Inventors: Florencio E. Hernandez, Orlando, FL (US); Andres Campiglia, Orlando, FL (US)

(73) Assignee: University of Central Florida Research Foundation, Inc., Orlando, FL (US)

(21) Appl. No.: 14/336,588

(22) Filed: Jul. 21, 2014

Related U.S. Application Data
(63) Continuation of application No. 11/843,227, filed on Aug. 22, 2007, now abandoned.
(60) Provisional application No. 60/839,824, filed on Aug. 24, 2006.

(51) Int. Cl. G01N 33/20; C02F 1/00; G01N 21/49; B01D 15/10; G01N 21/33; G01N 21/31

(52) U.S. Cl. CPC G01N 33/203; G01N 21/33; G01N 21/31; G01N 21/49; B01D 15/10; C02F 1/001; G01N 2201/061; G01N 2201/0696; G01N 2201/08; C02F 2101/20
USPC 210/295; 356/436

(57) ABSTRACT

A method of sensing Hg and related Hg sensing system for fluid samples includes the steps of providing a sensing solution including a plurality of anisotropic Au nanoparticles, and contacting a water sample or an air sample suspected of containing Hg or a vapor stream derived from the water sample with the plurality of anisotropic Au nanoparticles. A gold amalgam compound is generated when Hg is present in the sample. The presence, and optionally the concentration, of Hg in the sample are then determined using an optical method based on a change in at least one of absorption, reflectance and scattering of the solution. In a related inventive embodiment a filter for water treatment and remediation including the removal of Hg includes a first flow through grid having a Hg reducing material thereon on an inlet side of the filter and at least one flow through second grid including a surface having amalgamating material downstream from the first grid.

### CROSS REFERENCE TO RELATED APPLICATIONS/INCORPORATION BY REFERENCE STATEMENT

The present application is a continuation of U.S. application Ser. No. 11/843,227, filed Aug. 22, 2007, which claims the benefit under 35 U.S.C. 119(e) of U.S. Provisional Application No. 60/839,824, filed Aug. 24, 2006. The entire disclosures of both applications are hereby expressly incorporated herein by reference.

### FIELD OF THE INVENTION

The invention is related to Hg sensors and remediation, more particularly to metal nanoparticle-based Hg sensors and remediation.

### BACKGROUND OF THE INVENTION

Mercury (Hg) is a known environmental pollutant routinely released in gaseous form from power plants burning fossil fuels. According to the Environmental Protection Agency (EPA), coal-burning power plants are the largest human-caused source of Hg emissions to the air in the United States.

Hg release into the environment is problematic. Once released into the air, Hg eventually settles into water or onto land where it is washed. Hg is unique among metals in that it can evaporate when released to water or soil. Also, microbes can convert inorganic forms of Hg to organic forms which can be accumulated by aquatic life. For example, once in the water, certain bacteria change the Hg (inorganic) into methylmercury (an organic), which is absorbed by fish and transferred to animals that eat the fish, such as birds, bears, or humans. Because human exposure to high Hg levels is known to cause harm to the brain, heart, kidneys, lungs, and immune system of people, it is important to monitor Hg levels, particularly in bodies of water. Moreover, Hg is known to be especially damaging to unborn children, who can develop permanent mental problems from exposure to Hg while in the womb.

Currently, it is difficult to detect Hg in contaminated water. The available detection equipment is bulky and cannot detect small amounts of the pollutant. Several methods/systems exist to monitor concentration levels of Hg in water samples. Established techniques including Atomic Absorption Spectroscopy (AAS), Gas Chromatography-Inductively Coupled Plasma-Mass Spectrometry (GC-ICP-MS), Atomic Fluorescence Spectrometry (AFS), Inductively Coupled Plasma—Atomic Emission Spectrometry (ICP-AES), and reversed-phase High Performance Liquid Chromatography (HPLC), generally provide limits of detection at the parts-per-billion level. Their sensitivity, however, is achieved at the expenses of elaborate and time-consuming sample preparation and pre-concentration procedures.

The Environmental Protection Agency (EPA) method 1631 is one example. Prior to Hg detection by cold vapor AFS, Hg in the sample—which includes, but is not limited to, Hg(H), Hg(0), strongly organo-complexed Hg(II) compounds, adsorbed particulate Hg, and several covalently bound organo-mercurials such as CH₃HgCl, (CH₃)₂Hg, and C₆H₅HgOOCCH₃—is oxidized to Hg(II) and then reduced to volatile Hg(0). The vapor is then carried into the atomic fluorescence spectrometer via purge and trap with two high-surface area gold (Au) “traps” (usually Au-coated sand).

As a tentative means of reducing analysis time and cost, on-site sensing approaches capable to provide real-time Hg determination have been actively pursued. These include optical test strips, remote electrochemical sensors, ion-selective electrodes, fluorescence based sensor membranes, and piezoelectric quartz crystals. Although these approaches provide low detection limits and fast response times, they still lack the procedural simplicity for on-site analysis.

Moreover, improved Hg remediation and filtration systems are needed. The current EPA standard referred to as the Maximum Contaminant Level (MCL) is 2 ppb for Hg. One known way to remove Hg from drinking water is to use costly reverse osmosis systems. Such systems are electricity consuming, not adapted for large volume (e.g. public water supply) use, and are expensive even for consumer use. What is needed is a low cost system that does not require electrical power to operate to remove Hg from Hg-polluted water.

### SUMMARY

A method of sensing Hg for fluid samples comprises the steps of providing a sensing solution including a plurality of anisotropic Au nanoparticles, and contacting a water sample, and air sample, or a vapor stream derived from the water sample with the sensing solution. A gold amalgam compound is generated when Hg is present in the sample. The presence, and optionally the concentration, of Hg in the sample are then determined using an optical method based on a change in at least one of absorption, reflectance and scattering of the anisotropic Au nanoparticles resulting from the contacting step.

The change can comprise a shift in a maximum absorption wavelength. In this embodiment, the shift can comprise a shift in a longitudinal mode band of the anisotropic Au nanoparticles. The anisotropic Au nanoparticles can comprise Au nanorods. In one embodiment, the Au nanorods include surfactant along their length. An average aspect ratio (AR) of the anisotropic Au nanoparticles can be between 1.4 and 1.8. The method can further include the step of determining a concentration of Hg in the sample. For sensing of water samples the sensing solution generally includes at least one reducing agent capable of reducing Hg cations into elemental Hg.

A Hg sensing system comprises a sensing solution including a plurality of anisotropic Au nanoparticles, a light source directing incident light at the solution, a photodetector for detecting light emanating from the solution, and a processor connected to the photodetector for determining the presence of Hg in a fluid sample suspected of including Hg from data obtained from the photodetector after contacting the anisotropic Au nanoparticles with the sample. A spectrophotometer can be used to provide the light source and photodetector. An average AR of the anisotropic Au nanoparticles can be between 1.1 and 2.0. The solution can include at least one reducing agent capable of reducing Hg cations into elemental Hg.

A filter for water treatment and remediation including the removal of Hg comprises a housing including an inlet and an outlet, at least one flow through first grid having a reducing material capable of reducing Hg cations into elemental Hg thereon on an inlet side of the filter, and at least one flow through second grid including a surface comprising amalgamating material, wherein Hg in water to be filtered is reduced by the reducing material and removed from the water upon amalgamation with the amalgamating material. At least one of the first and second grids is porous grids. In one embodiment, the amalgamating material comprises Au, such as in the form of Au nanoparticles.

### BRIEF DESCRIPTION OF THE DRAWINGS

A fuller understanding of the present invention and the features and benefits thereof will be obtained upon review of the following detailed description together with the accompanying drawings, in which:

FIG. 1 shows a filter for water treatment and remediation including the removal of Hg, according to an embodiment of the invention.

FIG. 2 is a UV-V absorption spectrum of Au nanorods in nanopure water (solid line) and in 1.67×10⁻³ Mol/L NaBH₄ (dotted line) according to an embodiment of the invention.

FIG. 3 is a plot showing the wavelength shift of the longitudinal (N) and transversal (A) modes of Au nanorods as a function of NaBH₄ concentration according to an embodiment of the invention.

FIG. 4 is a UV-Vis absorption spectra showing the spectral shift at several Hg(II) concentrations according to an embodiment of the invention. The concentration range between 1.6×10⁻¹¹M and 6.3×10⁻¹¹M shows the spectra within the linear dynamic range of the calibration curve. The remaining spectra show the overlapping between the longitudinal and transversal absorption bands at higher Hg(II) concentrations.

FIG. 5 is a schematic showing the amalgamation of Hg with Au nanorods by the absorption of the Hg at the end of a nanorod to reduce the aspect ratio of the Au nanorod from 1.6 to 1.4, 1.2 and 1.0 according to an embodiment of the invention.

FIG. 6 shows scanned TEM and EDX images of Au nanorods in the absence and the presence of Hg according to an embodiment of the invention. row I=no Hg; row II=1.25×10⁻⁵M and row III=1.57×10⁻⁴ M Hg²⁺. All solutions were prepared in 1.67×10⁻³ Mol/L NaBH₄.

FIG. 7 is a plot showing the first derivative of the UV-Vis absorption spectrum of Au nanorods in nanopure water and Au nanorods in tap water according to an embodiment of the invention. First derivatives were taken over the peak of the longitudinal absorption band. Average peak in nanopure water was 599.72±0.01 nm and in tap water was 599.64±0.01 nm.

### DETAILED DESCRIPTION

A method of sensing mercury (Hg) in fluid samples comprises the steps of providing a sensing solution including plurality of anisotropic gold (Au) nanoparticles and contacting a water sample, an air sample, or a vapor stream derived from the water sample with solution having the plurality of anisotropic Au nanoparticles. A gold amalgam compound is generated on the nanoparticle surface when Hg is present in the sample. The presence, and optionally the concentration, of Hg in the sample are then determined using an optical method based on a change in at least one of absorption, reflectance and scattering of the anisotropic Au nanoparticles resulting from the contacting.

The change can be a shift in a maximum absorption wavelength, and can be associated with a blue shift in the maximum absorption wavelength of the longitudinal mode of the Au nanoparticles. Significantly, anisotropic particles, such as Au nanoparticles having an aspect ratio (AR) of 1.6 to 1.8, have been found by the inventors to provide both transversal mode (about 520 nm) and longitudinal mode (about 612 nm) absorption data, unlike spherical particles which provide only transversal mode absorption bands. For example, the AR is the length/diameter for a nanorod, or the area to thickness ratio for a nanoplate for the particles of the invention. As used herein an anisotropic nanoparticle is any nonspherical particle with a large dimension less than 1,000 nm that displays a transversal mode and a longitudinal mode in absorption spectra. The Inventors have also found that longitudinal mode band data provides significantly higher detection sensitivity as compared to transversal mode data.

El-Sayed et al. J. Phys. Chem. B 1999, 103, 3073-3077 provides a theoretical foundation to explain the occurrence of the two absorption bands that are typically observed in the UV-vis absorption spectra of Au nanorods. The two absorption bands correspond to the transversal and longitudinal modes of SPR. The transversal mode band belongs to the SPR along the short axis of the rod and appears at a shorter absorption maximum than the longitudinal mode band. The maximum absorption wavelength of the longitudinal mode—which corresponds to the SPR along the long axis of the rod—presents a linear correlation with the aspect ratio (length/diameter) of the nanorod. As the aspect of the nanorod or other anisotropic nanoparticle increases, the longitudinal mode band has been found by the present inventors to shift to longer wavelengths. The same behavior has been observed as the dielectric constant of the medium increases.

Related to absorption coefficient, the extinction coefficient may also be used. The extinction coefficient is (k) related to the absorption coefficient (α) by the expression k=λα/4π where λ is the wavelength of the incident radiation in a vacuum.

In one embodiment of the invention, the method of sensing Hg comprises providing a sensing solution including a plurality of anisotropic Au nanoparticles and at least one reducing agent capable of reducing Hg cations, and contacting a sample with the sensing solution. A gold amalgam compound is formed on the nanoparticle tip surface when Hg is present in the sample.

As noted above, the sample can be an air sample. In the air, Hg is generally elemental Hg and therefore can be detected directly (without reduction) by passing an air stream through the sensing solution. Air sensing according to the invention is expected to be useful to determine Hg migration through air currents in the atmosphere.

As used herein, anisotropic refers to particle shapes other than spherical that provide an AR of at least 1.1. The AR is the length/diameter for a nanorod, or the area to thickness ratio for a nanoplate for the particles of the invention. In the case of nanorods it has been found that the higher the AR of the nanoparticles, the better the dynamic range. However, for AR generally >2, sensitivity has been found to be reduced. Accordingly, for nanorods, ARs can be in the range from 1.2 to 2.0, most preferably between 1.4 and 1.8. The nanoparticles generally have a longest dimension from 5 to 250 nm in size. However, nanoparticles can have a longest dimension from 1 to 999 nm. In one embodiment, the Au nanoparticles include surfactant along their length. The surfactant present is generally associated with the formation of the nanoparticles. The presence of surfactant can improve measurement sensitivity.

Although Hg sensing according to the invention is described using Au nanoparticles, other metal nanoparticles that form an amalgam with Hg may be used, or in combination with Au nanoparticles. For example, Sn and Ag may also be used. However, unlike Au, due to the tendency metals such as Sn and Ag to oxidize, Au nanoparticles generally preferred.

When used, the reducing agent can be essentially any reducing agent capable of reducing Hg cations to elemental Hg. Although the reducing agent described herein in the Examples provided is NaBH₄, the invention is not so limited. Compounds other than NaBH₄ can be provided for Hg sensing in solutions. A table of reducing potentials can be used to identify generally suitable metals that can be used to reduce Hg cations to elemental Hg, such as for vapor sensing or for water filters according to the invention described below. For example, iron (Fe); copper (Cu) and silver (Ag) are exemplary metals that may be used to reduce Hg.

As noted above, in one embodiment of the invention, the concentration of Hg is determined based upon a change in the maximum absorption wavelength of anisotropic Au nanoparticles, such as Au nanorods. The selectivity and sensitivity of the present method provides a unique way to determine Hg in water samples without previously required separation and/or pre-concentration of the original sample.

The specificity towards Hg may result from the ability to amalgamate Hg with Au. Sample pre-concentration is not necessary due to the ultra-low limit of detection, e.g. 6.6×10⁻¹³ g/L demonstrated in the Examples below. Although not needed to practice the present invention, Applicants not seeking to be bound to the theory presented, present the following explanation for the high sensitivity provided by the invention.

The low limit of detection is attributed mainly to two main factors. The main contribution results from the high sensitivity of the surface plasmon resonance (SPR) of Au nanorods towards Hg amalgamation. A blue shift of the maximum absorption wavelength of the longitudinal mode band of Au nanorods is observed even in the presence of ultra-low traces of Hg. The direct correlation that was found by the Inventors to exist between wavelength shift and Hg concentration makes quantitative analysis simple. The second main reason is related to the outstanding precision of wavelength measurements. The limit of detection is based on the reproducibility of the maximum absorption wavelength of the reference signal (λ_{R}±s_{R}). Because the intensity of the reference signal utilized can provide a high signal-to-noise ratio of approximately equal to 70, or more, the contribution of instrumental noise to the reproducibility of measurements is negligible.

The procedure required to practice the invention is generally quite simple and it generally takes no longer than 10 minutes per sample. For liquid phase detection, solution mixing can be done in a test tube, which is well suited for on-site analysis with any portable spectrometer having appropriate spectral resolution.

In another embodiment of the invention, a sensing solution is not required. In this alternate embodiment, the plurality of anisotropic Au nanoparticles is secured to a surface of an optically transparent substrate, such as glass for visible light spectral applications. The nanoparticles in one embodiment are generally spaced apart from one another. In this embodiment the method can comprise step of bubbling a carrier gas (e.g. N₂) through a liquid sample to generate a vapor stream derived from the water sample which is directed to the nanoparticle coated substrate. The optically transparent substrate can comprises an optical fiber where the nanoparticles are secured to the end of the fiber.

Another embodiment of the invention is a new way to filter and reclaim Hg contaminated water. In this embodiment, Au or other nanoparticles capable of amalgamation with Hg are used to produce water filters for both large and small scale applications. Filters according to this embodiment of the invention can be fitted in applications including drainage systems that lead to waterways, residential sinks, and power plants.

A method of trapping Hg comprises the steps of providing a sensing solution including a plurality of Au nanoparticles and at least one reducing agent capable of reducing Hg cations. The sample is then mixed with the sensing solution, wherein a gold amalgam compound is formed when Hg is present in the sample.

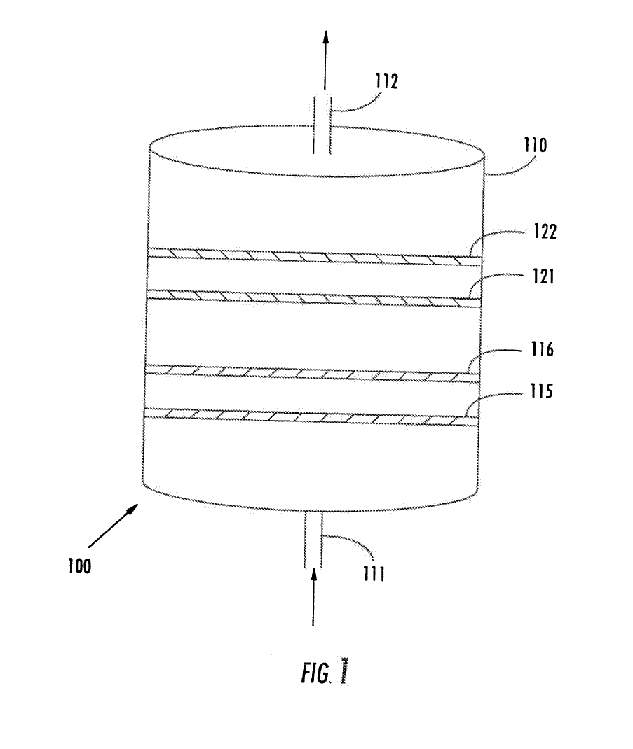

Filters for water treatment and remediation including the removal of Hg are also provided by the invention. In one embodiment, the filters are point-of-use filters. Referring to FIG. 1, a filter 100 according to an embodiment of the invention is shown. Filters include housing 110 having an inlet 111 and an outlet 112. Inside the housing is a reducing agent comprising Fe grids 115 and 116 and an amalgamation agent comprising Au (or Au coated) grids 121 and 122 downstream from the reducing agent. Porous grids 115, 116, 121, and 122 provide the preferred flow-through property. The grids can be porous or provide flow-through based on a plurality of holes that allows a solution to flow through the holes. Although grids 115 and 116 are shown in FIG. 1, in another embodiment, unbound reducing particles and/or amalgamation particles are disposed between porous separation plates (not shown) which are large enough to provide water flow therethrough, but small enough to prevent passage of the reducing and/or amalgamation particles.

Filter 100 not only removes Hg, but can also break down other harmful materials, such as organic materials, through the catalytic properties of Au nanoparticles. The catalytic properties of Au are enhanced using nanoparticles generally having a size less than about 15 nm.

In a residential embodiment, filters according to the invention are adapted to fit under the sink. Filters based on the invention may also be one stage of a multi-stage filter. In operation, water from a public water supply or well is passed through a filter according to the invention. If present, Hg ions are reduced by the Fe or other suitable metal on the grid. The elemental Hg then becomes bound to the Au grid through an amalgamation reaction.

Filters can be discarded after a predetermined period of time or based on water flow. Alternatively, filters according to the invention can be regenerated by a suitable heating process sufficient to evaporate Hg, such as heating to a temperature of about 350 to 360° C.

### EXAMPLES

It should be understood that the Examples described below are provided for illustrative purposes only and do not in any way define the scope of the invention.

### Chemicals

Analytical-reagent grade chemicals were used in all experiments. Acetone (C₃H₆O), tetradodecylammonium bromide (TDAB), cyclohexane (C₆H₁₂), hexadecyltrimethylammonium bromide (CTAB) (C₁₉H₄₂BrN), hydrogen tetrachloroaurate III (HAuCl₄), silver nitrate (AgNO₃), and ammonium fluoride (NH₄F) were obtained from Sigma-Aldrich. Hg II chloride (HgCl₂), sodium borohydride (NaBH₄), sodium nitrate (NaNO₃), lead II nitrate [Pb(NO₃)₂], barium acetate [Ba(C₂H₃O₂)₂], copper sulfate (CuSO₄), arsenic pentoxide (As₂O₅), chromic nitrate [Cr(NO₃)₃], and sodium chloride (NaCl) were purchased from Fisher Chemicals. All water used was obtained from a Barnstead Infinity ultrapure water system.

### Synthesis of Au Nanorods

Nanorods were synthesized according to the photochemical method developed by Kim et al. J. Am. Chem. Soc. 2002, 124, 14316-7. Briefly, 3 ml of a 0.08 M hexadecyltrimethylammonium bromide (CTAB) and 0.42 mg/ml aqueous solution of tetradodecylammonium bromide were mixed with 0.25 ml of a 0.024 M of chloroauric acid tri-hydrated (HAuCl₄•3H₂O) solution. To the mixture was added 0.065 ml of acetone and 0.045 ml of cyclohexane followed by 31.5 μl of a 0.01 M AgNO₃ aqueous solution. The final mixture was then irradiated for 24 hours with a 254 nm UV-light (420 mW/cm²). The appearance of a blue solution indicated nanorod formation. Au nanorods formed using this process include surfactant (here CTAB) along their length.

### Instrumentation

Absorbance measurements were carried out with a single-beam spectrophotometer (Model Cary 50, Varian) equipped with a 75 W pulsed Xenon lamp, 2 nm fixed band-pass and 24,000 nm.min⁻¹ maximum scan rate. Instrumental performance was monitored daily with a commercial standard purchased from Photon Technology International. The standard consists of a single crystal of dysprosium-activated yttrium aluminum garnet mounted in a cuvette-sized holder with well-characterized quasi-line absorption spectrum. Wavelength accuracy was monitored by comparing the position of several atomic lines to the maximum wavelengths provided by the manufacturer. Table 1 below summarizes the typical precision of measurements obtained within two hours of instrumental use.

TABLE 1
Instrumental Performance of Cary 50 Spectrophotometer^{a}

| Wavelength (nm) | Absorption Intensity (r.u.)^{b} |
|---|---|
| 352.00 ± 0.00 | 0.40700 ± 0.00100 |
| 366.00 ± 0.00 | 0.33400 ± 0.00100 |
| 447.00 ± 0.00 | 0.08800 ± 0.00100 |
| 752.00 ± 0.00 | 0.10178 ± 0.00100 |

^{a}Measurements were done with a commercial standard consisting of a single crystal of dysprosium-activated yttrium aluminum garnet mounted in a cuvette-sized holder. Reported values are the average of results extracted from 10 absorption spectra.
^{b}r.u. = relative units.

Results confirm the performance of the spectrometer according to the invention. More importantly, it was demonstrated that the spectrophotometer's response did not significantly affect the precision of measurements of experimental results presented herein.

For transmission electron microscopy (TEM) a FEI Tecnai F30 microscope was used operating at an accelerating voltage of 300 kV. This electron microscope has a field emission electron source and a super twin objective lens to yield a point-to-point resolution of 2 Angstrom. For the compositional analysis with energy dispersive x-ray spectroscopy (EDX) the sample was tilted 15 Degrees towards the detector. For TEM sample preparation drops of solution were deposited and dried on a standard TEM copper grid with amorphous carbon film.

### Spectra of Au Nanorods

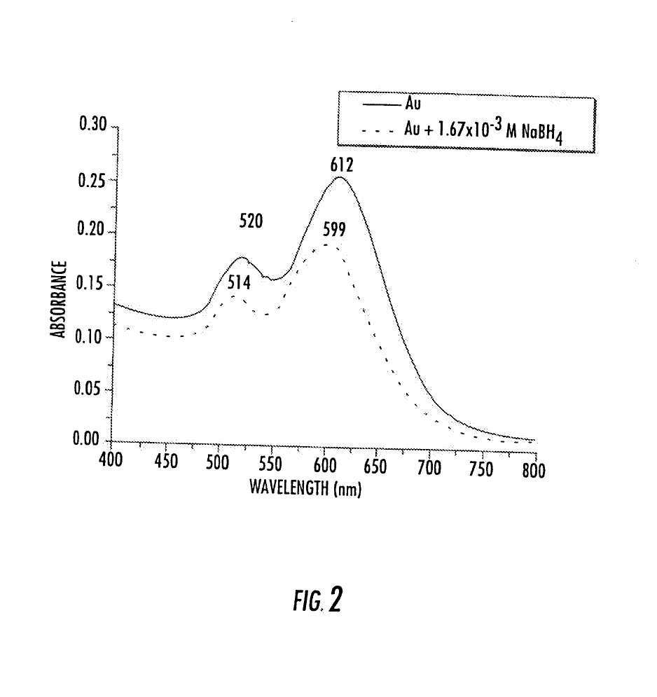

In the experiment performed Au nanorods were used having an average aspect ratio of 1.6. The solid line in FIG. 2 shows a typical absorption spectrum recorded from a nanorod suspension in pure water. Clearly, the absorption maxima of the transversal and longitudinal modes appear at 520 nm and 612 nm, respectively.

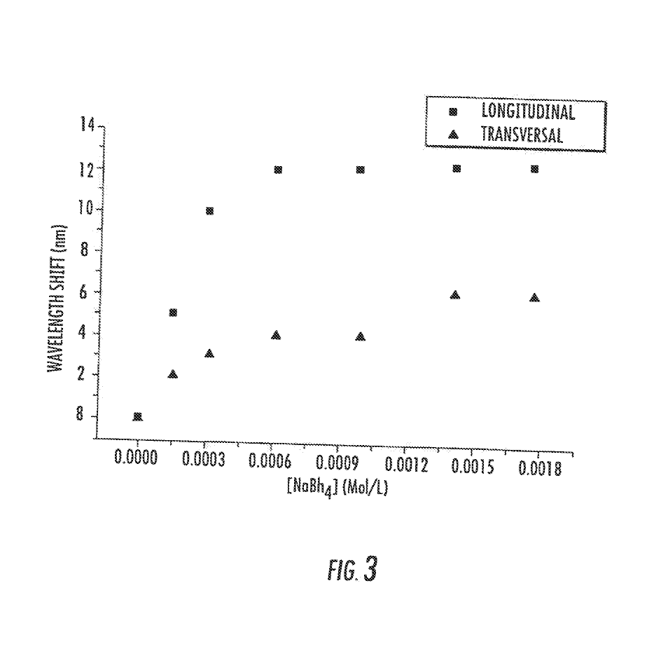

The first step in the process can comprise reducing all existing forms of oxidized Hg (e.g. Hg⁺² to Hg(0)). This was accomplished by mixing the water sample with a reducing agent, such as NaBH₄. This strong reducing agent is also capable to reduce any oxidized form of Au into Au(0). The broken line in FIG. 2 depicts the absorption profile of a nanorod suspension prepared in 1.67×10⁻³M NaBH₄. Comparison of the two spectra in FIG. 2 shows a blue shift (Δλmax) in the absorption maxima of the transversal (Δλmax=6 nm) and the longitudinal (Δλmax=12.1 nm) modes upon reduction by NaBH₄. Monitoring of the spectral shift as a function of NaBH₄ concentration yielded the plot in FIG. 3. The wavelength shifts experienced by the two modes reach plateaus at different NaBH₄ concentrations. The longitudinal mode was found to reach a maximum at 6.0×10⁻⁴ M, whereas the transversal mode reaches a maximum at 1.5×10⁻³ M. In addition, the absorption maximum of the longitudinal mode band shifts approximately 2× longer than the one corresponding to the transversal mode band. This is consistent with the higher sensitivity the longitudinal mode band towards the chemical environment of the nanorod.

The observed blue shift is attributed to the presence of un-reacted Au³⁺ in the nanorod suspension prior to the addition of NaBH₄. The reduction of Au³⁺ to Au(0), followed by its deposition on the surface of the nanorods, augment the size of the nanorods and can decrease the density of CTAB molecules covering their surface. Because the dielectric constant of the medium perceived by the nanorods is reduced, the maximum absorption bands of the two modes shift to shorter wavelengths. Experimental evidence supporting this assumption was obtained via TEM analysis where the nanorod after exposure to NaBH₄ solution displays an aspect ratio of 1.6 as opposed to the ratio of 1.8 for the nanorod before exposure to the NaBH₄.

### Effect of Hg (II) in the Absorption Spectrum of Au Nanorods

To assure complete reduction of oxidized Hg present in a water samples, all further studies were performed with a stoichiometric excess of reducing agent (1.67×10⁻³M NaBH₄). The absorption spectrum of Au nanorods was observed over a period of 8 hours and no further wavelength shifts occurred to the Au nanorod suspension to assure a reliable spectrum for reference purposes such that aspect ratio changes due to reduction of Au ions does not lead to systematic errors in the analysis of Hg in water.

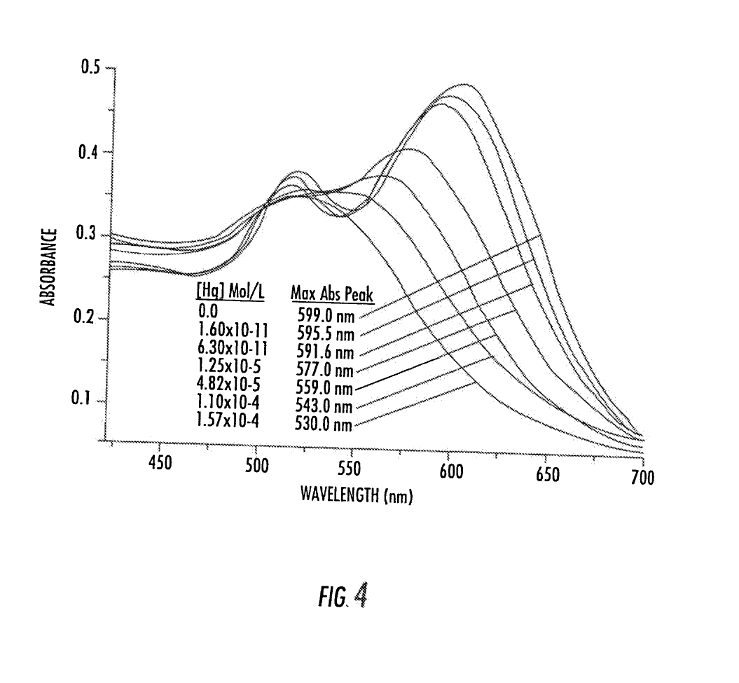

FIG. 4 shows the typical spectral shifts observed in the presence of Hg(II). The water samples were prepared by adding incremental volumes (1.0, 10.0 and 20.0 μL) of an aqueous standard 10⁻⁹ M HgCl₂ solution to a constant volume (600 μL) of a 1.64×10⁻⁵M Au nanorod solution in 1.67×10⁻³M NaBH₄. The concentration of nanorods was estimated assuming a 100% synthetic yield. All the spectra were recorded after 4 minutes of HgCl₂ addition, i.e. the minimum time at which a plateau was reached and no further spectral shifts were observed. The blue shift of the maximum absorption wavelength of the longitudinal band shows a direct correlation with Hg (II) concentration. The presence of Hg (II) at higher concentration ranges causes an overlap between the longitudinal and the transversal absorption bands of the nanorods. At [HgCl₂]=1.57×10⁻⁴ M, only one absorption band is observed. This evidence suggests the conversion of Au nanorods into Au nanospheres since Au nanospheres provide only a transversal absorption band.

Although not to be bound by a mechanism which accounts for the observed spectral phenomena, the chemical reactions believed to be involved that lead to the absorption band shifts include:

HgCl₂(aq)+2NaBH₄(aq)+6 H₂O→Hg(l)+7H₂(g)+2B(OH)₃(aq)+2NaCl(aq)

Hg(l)+3Au(s)→Au₃Hg (Low Hg atomic % content<30)

2Hg(l)+Au(s)→AuHg₂(l) (High Hg atomic % content>30)

In the presence of liquid Hg, two probable mechanisms of action can be considered to be operable, these mechanisms being surface covering of the nanorods by the liquid metal and/or amalgamation of the Au and Hg. Because Hg increases the effective dielectric constant of the medium perceived by the nanorods, the first possibility should result in the displacement of the longitudinal band toward longer wavelengths. The surface coating of the nanorods caused by the presence of Hg should dislocate the structure of the surrounding micelles that prevent the nanorods from aggregation and precipitation. As a result of Au dissolution, the second possibility—i.e. amalgamation—could cause a reduction of the effective aspect ratio of the nanorods and a blue shift of the maximum absorption wavelength of the longitudinal mode band could occur.

The data of FIG. 4 is consistent with a main mechanism of action involving amalgamation. When the active sites on Au nanorods mainly occur at the tips of the nanostructures, amalgamation should take place more efficiently on the tips of the nanorods and, therefore, it should decrease the effective ratio of the Au particle. Preferential amalgamation at the tips of the nanorods would also be favored by the presence of surfactant (CTAB) on the lateral sides of the nanorods. Their shielding effect restricts the amalgamation of Hg on the lateral walls of the nanorods. The fact that no further appreciable change is observed in the SPR of Au nanospheres beyond [HgCl₂]=1.57×10⁻⁴ M is consistent with its lack of sensitivity to particle size changes between about 3 and 60 nm.

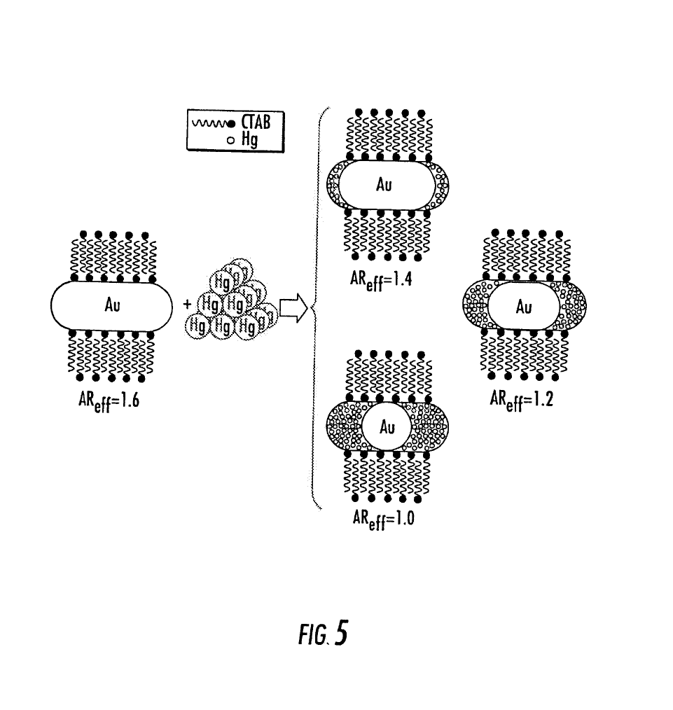

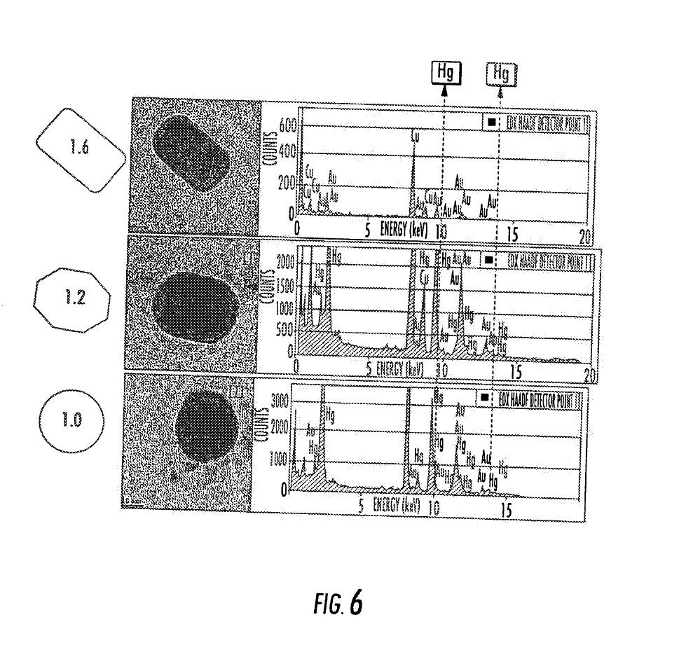

FIG. 5 is a schematic representation of shape changes of the Au particle to smaller ARs ranging from 1.0 to 1.4 due to preferential amalgamation at the tips of the Au nanorod where surfactant (CTAB) is attached to the Au nanoparticles. The experimental evidence for the schematic model of FIG. 5 is shown in FIG. 6. TEM images in the middle column of FIG. 6 show the shape and the AR of Au nanorods, represented as an idealized 2D projection in column 1, in the absence Hg²⁺ (I top row) and the presence of 1.25×10⁻⁵ M (II middle row) and 1.57×10⁻⁴ M (DI bottom row) Hg²⁺ solutions in the presence of 1.67×10⁻³M NaBH₄. As the concentration of Hg²⁺⁶⁰ increases as indicated in the along with EDX plots of FIG. 6, the aspect ratio decreases to the point at which the shape of the nanoparticles becomes spherical. The changes in aspect ratio and shape of Au nanorods correlates to the changes observed in their absorption spectra. The blue shift of the longitudinal band observed in the absorption spectra in the presence of 1.25×10⁻⁵M Hg²⁺ can be attributed to the change in aspect ratio. Similarly, a single absorption band observed in the presence of 1.57×10⁻⁴M Hg²⁺ solution can be attributed to the spherical shape of the nanoparticles. Additional experimental evidence of amalgamation is provided by EDX analysis. As expected, the Hg content in the nanoparticles is inversely proportional to their aspect ratio. Spherical nanoparticles contain more Hg than nanorods.

### Analytical Figures of Merit

The limit of detection (LOD=x_{L}) of an analytical method can be calculated by x_{L}=x_{B}+ks_{B}, where x_{B} is the main average of blank responses, s_{B} is the standard deviation of blank measurements, and k is a numerical factor chosen in accordance to the desired confidence level. Therefore, the best LOD is usually obtained with the minimum blank signal value. Because the inventive approach is based on absorption spectral shifts upon Hg interaction with Au nanorods, the LOD provided also depends on the reproducibility (s_{R}) of the reference wavelength (λᵣ), i.e. the maximum absorption wavelength of Au nanorods in the absence of Hg. In the lack of matrix interference, the main source of wavelength variation is instrumental noise. The extent to which instrumental noise deteriorates s_{R} depends on the magnitude of the reference signal. Because the magnitude of the reference signal is directly proportional to the concentration of Au nanorods, one can adjust this parameter to optimize λ_{R} reproducibility. In doing so, it should be kept in mind that there is a direct correlation between Au nanorods and Hg concentration and that Hg traces are detected only with low nanorods concentrations. Careful investigation of these parameters led the Inventors to set a 1.64×10⁻⁵ M working concentration. Multiple runs (n=16) of this standard solution yielded an average maximum absorption peak at λ_{R}=599.72±0.02 nm. Each λ_{R} was calculated by taking the first derivative of each absorption spectrum between 560 and 640 nm. Keeping in mind the average intensity of the reference signal (0.05500±0.002 r.u.), comparison of λ_{R}±s_{R} to values in Table 1 shows no significant contribution from instrumental noise.

Table 2 (shown below) summaries the analytical figures of merit obtained with a 1.64×10⁻⁵M Au nanorods solution. Analytical figures of merit were obtained adding small aliquots of a 1×10⁻⁹ M Hg(II) solution to a 1.64×10⁻⁵M Au nanorods solution in 3.34×10⁻³M NaBH₄. Each intensity value plotted in the calibration graph is the average of six replicate measurements.

TABLE 2
Analytical Figures of Merit for
Hg Determination with Au Nanorods

| Linear Dynamic Range^{a} (g/L) | 1.98 × 10⁻¹²-3.11 × 10⁻⁸ |
|---|---|
| Limit of Detection^{b} (g/L) | 6.6 × 10⁻¹³ |
| Correlation Coefficient^{c} | 0.9998 |
| Relative Standard Deviation^{d} (%) | 0.33 |

^{a}Lower concentration limit was calculated as 3 × LOD.
^{b}Calculated using 3S_{R}/m, where S_{R} is the standard deviation of the blank (N = 16).
^{c}Each calibration curve was built on six different concentration levels, with three replicates for each level (N = 18).
^{d}RSD = (100 × S)/X, where S is the standard deviation of a concentration in the middle of the LDR and X is the average wavelength shift at that concentration.

The calibration curve was built plotting the wavelength shift of the first absorption derivative as a function of Hg²⁺ molar concentration. The wavelength shifts plotted in the calibration graph are the averages of individual measurements taken from three aliquots of the same working solution. The linear dynamic range of the calibration curve is based on at least six Hg concentrations. The correlation coefficient and the slope of the log-log plot are close to unity, demonstrating a linear relationship between Hg concentration and wavelength shift. The relative standard deviation at medium concentrations was below 1%, which shows excellent reproducibility of measurements. The LOD was calculated with the equation LOD=3s_{R}/m, where m is the slope of the calibration curve and s_{R}=±0.02 nm. The standard deviation was calculated based on 16 measurements of the reference signal (λ_{R}). Although a straightforward comparison with reported LOD is difficult because different instrumental setups and mathematical approaches have been used for their determination, it can be concluded that the LOD obtained using the invention (1.53×10⁻¹⁰ g/L) is at least one and three orders of magnitude better than those of previously reported with established techniques and with the most sensitive sensors, respectively.

### Hg Determination in Tap Water

Based on the Safe Water Drinking Act, all community water systems must comply with a set of standards established by the EPA. The maximum concentration of Hg allowed in drinking water is 2×10⁻⁹ g/L. Because this concentration is well above the LOD provided by the Invention, the invention was used for monitoring Hg in tap water. Several inorganic ions were first screened for potential interference as shown in Table 3 below.

TABLE 3
Effect of Inorganic Ions on the Maximum
Absorption Wavelength of Au Nanorods^{a}

| | | |
|---|---|---|
| Compound^{b} | Concentration^{c} | Au abs peak^{d} |

| | | | |
|---|---|---|---|
| As₂O₅ | 5 | ng · mL⁻¹ | 599.71 ± 0.02 |
| Ba(C₂H₃O₂)₂ | 36 | ng · mL⁻¹ | 599.72 ± 0.02 |
| Cr(NO₃)₃ | 2 | ng · mL⁻¹ | 599.72 ± 0.02 |
| CuSO₄ | 590 | ng · mL⁻¹ | 599.72 ± 0.02 |
| NH₄F | 1 | μg · mL⁻¹ | 599.71 ± 0.02 |
| NaCl | 29 | μg · mL⁻¹ | 599.72 ± 0.02 |
| NaNO₃ | 120 | ng · mL⁻¹ | 599.70 ± 0.02 |
| Pb(NO₃)₂ | 1 | ng · mL⁻¹ | 599.71 ± 0.0 |

^{a}All measurements were made using a 1.64 × 10⁻⁵M Au nanorods solution in 3.34 ×10⁻³M NaBH₄.
^{b}Compound dissolved in nanopure water.
^{c}Final concentration of ion in the nanorods aqueous solution.
^{d}Average wavelength and standard deviation of three replicate measurements of maximum absorption wavelength in the presence of potential interference. These values show no statistical difference when compared to the reference value in the absence of inorganic ions (599.72 ± 0.02).

The selected ionic compounds followed literature reports on typical tap water composition. Arsenic, barium, chromium, copper, fluoride, nitrate and lead are also part of the EPA pollutants standard list. The reference sample consisted of a nanopure water solution with 1.64×10⁻⁵M Au nanorods and 1.67×10⁻³ M NaBH₄. Each ion was individually tested by spiking the reference solution with a nanopure water solution of appropriate ion concentration. None of the ions caused noticeable spectral interference. The maximum absorption wavelength of the reference signal remained the same, i.e. λ_{R}=599.72±0.02 nm. Lack of interference was also observed in the presence of all the ions in solution.

Quantitative analysis was performed via the multiple standard addition method. The reference spectrum was recorded from a mixture of 5 mL of nanopure water with an equal volume of a stock reference solution containing 3.28×10⁻⁵M Au nanorods and 3.34×10⁻³ M NaBH₄. The spectrum corresponding to the zero standard addition was recorded from a mixture of 5 mL of tap water with an equal volume of stock reference solution. The standard additions were made to 5 mL aliquots of tap water. Each aliquot was spiked with microliters of a 1.5×10⁻⁹ M HgCl₂ standard solution. Their absorption spectra were recorded after mixing them with equal volumes of the stock reference solution. The least squares fitting of the standard addition curve provided a straight line (correlation coefficient=0.9975) best described by the equation Y=2.2634×10¹¹X+1.88754. Extrapolation to Y=0 provided a Hg concentration (1.67×10⁻⁹ g/L) in the tap water sample above our LOD and slightly below the maximum level allowed by the EPA.

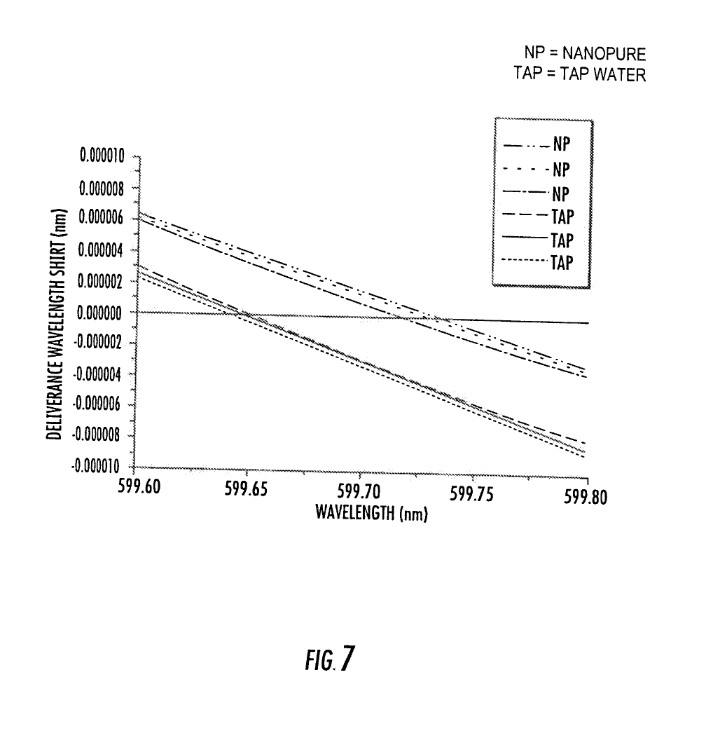

FIG. 7 compares the first derivatives of the longitudinal absorption bands recorded from three aliquots of nanopure and tap water. The maximum absorption wavelength of each aliquot was obtained at the intercept with the zero derivative wavelength shift. For three sample aliquots, the average maximum peak in nanopure water was 599.72±0.01 nm and in tap water was 599.64±0.01nm. Within a 95% confidence interval, these values are statistically different, which demonstrates the ability to determine trace levels of Hg accurately. Even at these low concentration levels, the relative standard deviation of the method was found to be excellent (<0.002%).

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the following claims.

We claim:

1. A method of sensing Hg in fluids, comprising the steps of:
providing a sensing solution including a plurality of anisotropic Au nanoparticles;
contacting a fluid sample suspected of containing Hg with said sensing solution; and
determining if Hg is present in said sample using an optical method, said optical method comprising detecting a change in an optical parameter resulting from said contacting step.

2. The method of claim 1, wherein, said change comprises a spectral change of at least one of absorption, reflectance and scattering.

3. The method of claim 1, wherein the step of determining includes quantification of said Hg in said sample.

4. The method of claim 1, wherein said change comprises a shift in a maximum absorption wavelength.

5. The method of claim 4, wherein said shift comprises a shift in a longitudinal mode band of said anisotropic Au nanoparticles.

6. The method of claim 1, wherein said fluid sample comprises a water sample, an air sample, or a vapor stream derived from said water sample.

7. The method of claim 1, wherein said anisotropic Au nanoparticles in said sensing solution comprise Au nanorods.

8. The method of claim 7, wherein said Au nanorods include surfactant along at least a portion of their length.

9. The method of claim 1, wherein an average aspect ratio of said Au nanoparticles in said sensing solution is between 1.4 and 1.8.

10. The method of claim 1, further comprising the step determining a concentration of said Hg in said sample.

11. The method of claim 1, wherein said solution includes at least one reducing agent operable to reduce Hg cations into elemental Hg.

12. A Hg sensing system, comprising:
a sensing solution including a plurality of anisotropic Au nanoparticles;
a light source directing incident light at said solution;
a photodetector for detecting light emanating from said solution, and a processor connected to said photodetector for determining the presence of Hg in a fluid sample suspected of including Hg from data obtained from said photodetector after contacting said anisotropic Au nanoparticles with said sample.

13. The system of claim 12, wherein a spectrophotometer provides said light source and said photodetector.

14. The system of claim 12, wherein an average aspect ratio of said anisotropic Au nanoparticles is between 1.1 and 2.0.

15. The system of claim 14, wherein an average aspect ratio of said anisotropic Au nanoparticles is between 1.4 and 1.8.

16. The system of claim 12, wherein said solution including at least one reducing agent capable of reducing Hg cations into elemental Hg.

17. A filter for water treatment and remediation including the removal of Hg, comprising:
a housing including an inlet and an outlet,
at least one flow through first grid having a reducing material capable of reducing Hg cations into elemental Hg thereon on an inlet side of said filter, and
at least one flow through second grid including a surface comprising amalgamating material, wherein Hg in water to be filtered is reduced by said reducing material and removed from said water upon amalgamation with said amalgamating material.

18. The filter of claim 17, wherein at least one of said first and second grid are porous grids.

19. The filter of claim 17, wherein said amalgamating material comprises Au.

20. The filter of claim 17, wherein said Au comprises Au nanoparticles.
